(19) United States
(12) Patent Application Publication
(10) Pub. No.: US 2026/0355284 A1
(43) Pub. Date: Nov. 12, 2026

(54) METHODS FOR INDUCING SATIETY AND TREATING METABOLIC DISORDERS

(71) Applicant: Gila Therapeutics, Inc., Minneapolis, MN (US)

(72) Inventor: Thomas VASICEK, Minneapolis, MN (US)

(21) Appl. No.: 18/577,326

(22) PCT Filed: Jul. 8, 2022
(86) PCT No.: PCT/US2022/036439

§ 371 Date: Jan. 8, 2024

Related U.S. Application Data
(60) Provisional application No. 63/219,407, filed on Jul. 8, 2021.

(51) Int. Cl. A61K 38/26; A61K 38/22; A61K 38/23; A61K 38/28

(52) U.S. Cl. CPC A61K 38/26; A61K 38/2264; A61K 38/23; A61K 38/28

(57) ABSTRACT

The invention provides methods for inducing satiety and treatment of metabolic disorders, diabetes, obesity, and obesity-related conditions. The methods include administration of a metabolic hormone via an intranasal, topical gastrointestinal, or intrarectal route.

### BACKGROUND OF THE INVENTION

The prevalence of obesity continues to increase worldwide. However, there is still a lack of effective, long-term, non-invasive treatments for obesity and other metabolism related disorders. Induction of satiety and treatment of metabolic syndrome, diabetes, obesity, and obesity-related disorders with existing therapeutics often does not produce suitable therapeutic effects. Accordingly, new treatments are needed.

### SEQUENCE LISTING

The instant application contains a Sequence Listing which has been submitted electronically in XML format and is hereby incorporated by reference in its entirety. Said XML copy, created on Jul. 15, 2026, is named “51547-016002_Sequence_Listing_7_15_26.XML” and is 21,512 bytes in size.

### SUMMARY OF THE INVENTION

In one aspect, the invention features a method for inducing satiety or treating a disease or disorder selected from a metabolic syndrome, obesity, an obesity-related disorder, diabetes, fatty liver disease, nonalcoholic steatohepatitis, chronic kidney disease, polycystic ovary syndrome, cardiovascular disease, obstructive sleep apnea, retinopathy, peripheral vascular disease, peripheral artery disease, and neuropathy (e.g., diabetic neuropathy) in a subject in need thereof. The method includes administering to the subject a composition that includes an agent selected from Peptide YY (PYY), glucagon-like peptide 1 (GLP-1), leptin, amylin, insulin, and calcitonin, or an analog, variant, or biologically active fragment thereof. The composition is administered intranasally, topically to the gastrointestinal (GI) tract, or intrarectally. The composition provides treatment without substantially changing the concentration of the agent in the blood of the subject.

In some embodiments, the composition is administered intranasally. The composition may be formulated, for example, as a spray, a semisolid, a particulate, or in a lipid-based carrier.

In some embodiments, the composition is administered topically to the GI tract. The composition may be formulated, for example, as a GI patch.

In some embodiments, the composition is administered intrarectally. The composition may be formulated, for example, as a suppository.

In some embodiments, the dose of the agent (e.g., PYY, GLP-1, leptin, amylin, insulin, or calcitonin, or an analog, variant, or biologically active fragment thereof) is from 1 ng to 20 mg per 100 kg body weight. For example, the dose of the agent may be from 1 ng to 10 ng per 100 kg body weight, e.g., 1 ng, 2 ng, 3 ng, 4 ng, 5 ng, 6 ng, 7 ng, 8 ng, 9 ng, or 10 ng per 100 kg body weight, e.g., from 10 ng to 100 ng per 100 kg body weight, e.g., 20 ng, 30 ng, 40 ng, 50 ng, 60 ng, 70 ng, 80 ng, 90 ng, or 100 ng per 100 kg body weight, e.g., from 100 ng to 1 μg per 100 kg body weight, e.g., 200 ng, 300 ng, 400 ng, 500 ng, 600 ng, 700 ng, 800 ng, 900 ng, or 1 μg per 100 kg body weight, e.g., from 1 μg to 10 μg per 100 kg, e.g., 2 μg, 3, μg, 4 μg, 5 μg, 6 μg, 7 μg, 8 μg, 9 μg, or 10 μg per 100 kg body weight, e.g., from 10 μg to 100 μg per 100 kg body weight, e.g., 10 μg, 20 μg, 30 μg, 40 μg, 50 μg, 60 μg, 70 μg, 80 μg, 90 μg, or 100 μg per kg body weight, e.g., from 100 μg to 1 mg per kg of body weight, e.g., 100 μg, 200 μg, 300 μg, 400 μg, 500 μg, 600 μg, 700 μg, 800 μg, 900 μg, or 1 mg per kg body weight, or e.g., from 1 mg to 20 mg per 100 kg body weight, e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg per 100 kg body weight.

In some embodiments, the composition includes PYY or an analog, variant, or biologically active fragment thereof. The PYY fragment may be, for example, PYY(3-36).

In some embodiments, the composition includes GLP-1 or an analog, variant, or biologically active fragment thereof.

In some embodiments, the composition includes leptin or an analog, variant, or biologically active fragment thereof.

In some embodiments, the composition includes amylin or an analog, variant, or biologically active fragment thereof.

In some embodiments, the composition includes insulin or an analog, variant, or biologically active fragment thereof.

In some embodiments, the composition does not include insulin or an analog, variant, or biologically active fragment thereof.

In some embodiments, the composition includes calcitonin or an analog, variant, or biologically active fragment thereof.

### DEFINITIONS

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

The term “metabolic disorder,” as used herein, refers to a human or animal condition or disease associated with and/or resulting from abnormal function or control of the metabolic system (e.g., obesity, diabetes, fatty liver disease, nonalcoholic steatohepatitis, polycystic ovary syndrome, elevated blood glucose levels, chronic kidney disease, cardiovascular disease, obstructive sleep apnea, retinopathy, neuropathy (e.g., diabetic neuropathy), peripheral artery disease, and/or peripheral vascular disease). The term “disorder” generally refers to disruption to regular bodily structure and function or a pathophysiological response to internal or external factors.

As used herein, the term “subject,” refers to a human or non-human animal (e.g., a mammal).

### DETAILED DESCRIPTION OF THE INVENTION

In general, the invention features methods for inducing satiety and treating a condition affecting metabolism, such as metabolic syndrome, diabetes, obesity, and obesity-related disorders. The invention is based, in part, upon the surprising discovery that administration of a metabolic hormone, such as PYY, via an intranasal, topical GI, or intrarectal route can activate a neural receptor in the central nervous system to effectively treat the disease or disorder.

By administering the metabolic hormone via one of these routes, the composition can provide treatment to the subject without substantially changing the concentration of the agent in the blood of the subject. Furthermore, these routes of administration avoid systemic administration, which are known to produce unwanted side effects, such as nausea, injection site pain, or malaise.

In general, by administering the metabolic hormone via a non-systemic pathway, the hormone can target specific pleasure centers in the brain, activate critical brain regions, and avoid neural or non-neural targets that can cause clinical risk (e.g., toxicity) or side effects, such as nausea. For example, systemic administration of a metabolic hormone activates neural receptors in the hypothalamus, nucleus tractus solitarius (NTS), and area postrema, whereas non-systemic routes of administration as described herein activates neural receptors in the hypothalamus and NTS, but substantially avoids the area postrema. By targeting neural receptors, e.g., in the nose, rectum, or GI tract, the neural receptors and connections can sufficiently activate the pleasure centers in the CNS, which control satiety. Accordingly, activation of these pleasure centers may provide treatment for a metabolic disorder or a satiety disorder associated with dysregulated pleasure centers in the brain. The methods are described in more detail below.

### Metabolic Hormone Compositions

The methods described herein include administration of a metabolic hormone or a biologically active fragment or variant thereof. The metabolic hormone targets (e.g., binds, associates, or interacts with) a Y2 receptor in the nasal cavity, GI tract, or rectum of a subject. In some embodiments, the metabolic hormone is PYY, PYY(3-36), leptin, amylin, insulin, calcitonin, or GLP-1, or a variant, analog, or biologically active fragment thereof.

In some embodiments, the dose of the metabolic hormone or a biologically active fragment, variant, or analog thereof is from 0.1 ng to 20 mg. For example, the dose of the agent may be from 0.1 ng to 1 ng, e.g., 0.2 ng, 0.3 ng, 0.4 ng, 0.5 ng, 0.6 ng, 0.7 ng, 0.8 ng, 0.9 ng, or 1 ng, e.g., 1 ng to 10 ng, e.g., 1 ng, 2 ng, 3 ng, 4 ng, 5 ng, 6 ng, 7 ng, 8 ng, 9 ng, or 10 ng, e.g., from 10 ng to 100 ng, e.g., 20 ng, 30 ng, 40 ng, 50 ng, 60 ng, 70 ng, 80 ng, 90 ng, or 100 ng, e.g., from 100 ng to 1 μg, e.g., 200 ng, 300 ng, 400 ng, 500 ng, 600 ng, 700 ng, 800 ng, 900 ng, or 1 μg, e.g., from 1 μg to 10 μg, e.g., 2 μg, 3, μg, 4 μg, 5 μg, 6 μg, 7 μg, 8 μg, 9 μg, or 10 μg, e.g., from 10 μg to 100 μg, e.g., 10 μg, 20 μg, 30 μg, 40 μg, 50 μg, 60 μg, 70 μg, 80 μg, 90 μg, or 100 μg, e.g., from 100 μg to 1 mg, e.g., 100 μg, 200 μg, 300 μg, 400 μg, 500 μg, 600 μg, 700 μg, 800 μg, 900 μg, or 1 mg, e.g., from 1 mg to 20 mg, e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg.

In some embodiments, the metabolic hormone is PYY or an analog, variant, or biologically active fragment thereof. In some embodiments, PYY or its variant, analog, or biologically active fragment thereof has at least 70% (e.g., 70%, 75%, 80%, 85%, 90%, 95%, 97%, 99%, or 100%) sequence identity to SEQ ID NO: 1. In some embodiments, PYY has the amino acid sequence set forth in SEQ ID NO: 1. In some embodiments, the methods described herein include administration of PYY or variant, analog, or biologically active fragment thereof in a dose of from about 2.5 μg to about 2.5 mg. In some embodiments, the methods described herein include administration of PYY or variant, analog, or biologically active fragment thereof in a dose of from about 10 μg to about 1 mg. In some embodiments, the methods described herein include administration of PYY or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the PYY fragment is PYY(3-36) or a variant, analog, or biologically active fragment thereof. In some embodiments, PYY(3-36) or its variant, analog, or biologically active fragment thereof has at least 70% (e.g., at least 70%, 75%, 80%, 85%, 90%, 95%, 97%, 99%, or 100%) sequence identity to SEQ ID NO: 2. In some embodiments, PYY(3-36) has the amino acid sequence set forth in SEQ ID NO: 2. In some embodiments, the methods described herein include administration of PYY(3-36) or variant, analog, or biologically active fragment thereof in a dose of from about 2.5 μg to about 2.5 mg. In some embodiments, the methods described herein include administration of PYY(3-36) or variant, analog, or biologically active fragment thereof in a dose of from about 10 μg to about 1 mg. In some embodiments, the methods described herein include administration of PYY(3-36) or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the PYY variant is [Pro34]PYY. In some embodiments, [Pro34]PYY or its variant, analog, or biologically active fragment thereof has at least 70% (e.g., at least 70%, 75%, 80%, 85%, 90%, 95%, 97%, 99%, or 100%) sequence identity to SEQ ID NO: 11. In some embodiments, [Pro34]PYY has the amino acid sequence set forth in SEQ ID NO: 11. In some embodiments, the PYY analog is NNC065-1273, which contains the PYY(3-36) polypeptide with a beta-homo-arginine at position 35. In some embodiments, the PYY analog is NNC0165-1875. In some embodiments, the PYY analog is NNC0165-1562. In some embodiments, the PYY analog is described, e.g., in Lear et al. J. of Med. Chem. 63:9660-9671, 2020, which is hereby incorporated by reference in its entirety. In some embodiments, the PYY analog is PYY-Ab and PYY conjugated to one or more PEG moieties, e.g., as described in Rangwala et al. Cell Metab. 29:837-843, 2019, which is hereby incorporated by reference in its entirety. In some embodiments, the PYY analog or variant is described, e.g., in U.S. Pat. No. 8,217,001, the disclosure of which is hereby incorporated by reference in its entirety.

In some embodiments, the PYY(3-36) variant, analog, or biologically active fragment thereof is PYY(26-36), PYY(25-36), PYY(24-36), PYY(23-36), PYY(22-36), PYY(21-36), PYY(20-36), PYY(19-36), PYY(18-36), PYY(17-36), PYY(16-36), PYY(15-36), PYY(14-36), PYY(13-36), PYY(12-36), PYY(11-36), PYY(10-36), PYY(9-36), PYY(8-36), PYY(7-36), PYY(6-36), PYY(5-36), or PYY(4-36), as in Balasubramaniam et al., Pept Res 1:32-35, 1998; Liu et al., J. Gastrointest Surg. 5:147-152, 2001, herein incorporated by reference in their entirety. In some embodiments, the PYY(3-36) variant, analog, or fragment thereof may be a fragment with a single point mutation e.g., single point mutation of PYY(25-36) such as [Lys²⁵]PPY(25-36), [Thr²⁷]PPY(25-36), [Phe²¹]PPY(25-36), [Ile²⁸]PYY(25-36), [Val²⁸]PYY(25-36), [Gln²⁹]PYY(25-36), [Ile³⁰]PYY(25-36), [Val³⁰]PYY(25-36), [Ile³¹]PYY(25-36), [Leu³¹]PYY(25-36), [Ser³²]PYY(25-36), [Lys³³]PYY(25-36), [Asn³⁴]PYY(25-36), [Lys³⁵]PYY(25-36), [Thr³⁶]PYY(25-36), or [Phe³⁶]PYY(25-36) or a single point mutation of PYY(24-36) such as [Ile²⁴]PYY(24-36) or [Val24]PYY(24-36). In some embodiments, the PYY(3-36) variant, analog, or biologically active fragment thereof may be a fragment with a double point mutation e.g., double point mutation of PYY(25-36) such as [Lys25, Thr27]PPY(25-36), [Lys25, Phe27]PPY(25-36), [Lys25, Ile28]PPY(25-36), [Lys25, Val28]PPY(25-36), [Lys25, Gln29]PPY(25-36), [Lys²⁵, Ile³⁰]PPY(25-36), [Lys²⁵, Val³⁰]PPY(25-36), [Lys²⁵, Ile³¹]PPY(25-36), [Lys²⁵, Leu³¹]PPY(25-36), [Lys²⁵, Ser³²]PPY(25-36), [Lys²⁵, Lys³³]PPY(25-36), [Lys²⁵, Asn³⁴]PPY(25-36), [Lys²⁵, Lys³⁵]PPY(25-36), [Lys²⁵, Thr³⁶]PPY(25-36), [Lys²⁵, Phe³⁶]PPY(25-36), [Thr²⁷, Ile²⁸]PPY(25-36), [Thr²⁷, Val²⁸]PPY(25-36), [Thr²⁷, Gln²⁹]PPY(25-36), [Thr²⁷, Ile³⁰]PPY(25-36), [Thr²⁷, Val³⁰]PPY(25-36), [Thr²⁷, Ile³¹]PPY(25-36), [Thr²⁷, Leu³¹]PPY(25-36), [Thr²⁷, Ser³²]PPY(25-36), [Thr²⁷, Lys³³]PPY(25-36), [Thr²⁷, Asn³⁴]PPY(25-36), [Thr²⁷, Lys³⁵]PPY(25-36), [Thr²⁷, Thr³⁶]PPY(25-36), [Thr²⁷, Phe³⁶]PPY(25-36), [Phe²⁷, Ile²⁸]PPY(25-36), [Phe²⁷, Val²⁸]PPY(25-36), [Phe²⁷, Gln²⁹]PPY(25-36), [Phe²⁷, Ile³⁰]PPY(25-36), [Phe²⁷, Val³⁰]PPY(25-36), [Phe²⁷, Ile³¹]PPY(25-36), [Phe²⁷, Leu³¹]PPY(25-36), [Phe²⁷, Ser³²]PPY(25-36), [Phe²⁷, Lys³³]PPY(25-36), [Phe²⁷, Asn³⁴]PPY(25-36), [Phe²⁷, Lys³⁵]PPY(25-36), [Phe²⁷, Thr³⁶]PPY(25-36), [Phe²⁷, Phe³⁶]PPY(25-36), [Gln²⁹, Ile³⁰]PYY(25-36), [Gln²⁹, Val³⁰]PYY(25-36), [Gln²⁹, Ile³¹]PYY(25-36), [Gln²⁹, Leu³¹]PYY(25-36), [Gln²⁹, Ser³²]PYY(25-36), [Gln²⁹, Leu³³]PYY(25-36), [Gln²⁹, Asn³⁴]PYY(25-36), [Gln²⁹, Leu³³]PYY(25-36), [Gln²⁹, Thr³⁶]PYY(25-36), [Gln²⁹, Phe³⁰]PYY(25-36), [Ile³⁰, Ile³¹]PYY(25-36), [Ile³⁰, Leu³¹]PYY(25-36), [Ile³⁰, Ser³²]PYY(25-36), [Ile³⁰, Lys³³]PYY(25-36), [Ile³⁰, Asn³⁴]PYY(25-36), [Ile³⁰, Lys³³]PYY(25-36), [Ile³⁰, Thr³⁰]PYY(25-36), [Ile³⁰, Phe³⁰]PYY(25-36), [Val³⁰, Ile³¹]PYY(25-36), [Val³⁰, Leu³¹]PYY(25-36), [Val³⁰, Ser³²]PYY(25-36), [Val³⁰, Lys³³]PYY(25-36), [Val³⁰, Asn³⁴]PYY(25-36), [Val³⁰, Lys³⁵]PYY(25-36), [Val³⁰, Thr³⁰]PYY(25-36), [Val³⁰, Phe³⁰]PYY(25-36), [Ile³¹, Ser³²]PYY(25-36), [Ile³¹, Lys³³]PYY(25-36), [Ile³¹, Asn³⁴]PYY(25-36), [Ile³¹, Lys³³]PYY(25-36), [Ile³¹, Thr³⁰]PYY(25-36), [Leu³¹, Phe³⁰]PYY(25-36), [Leu³¹, Ser³²]PYY(25-36), [Val³¹, Lys³³]PYY(25-36), [Leu³¹, Asn³⁴]PYY(25-36), [Leu³¹, Lys³³]PYY(25-36), [Leu³¹, Thr³⁰]PYY(25-36), [Leu³¹, Phe³⁰]PYY(25-36), [Ser³², Lys³³]PYY(25-36), [Ser³², Asn³⁴]PYY(25-36), [Ser³², Lys³⁵]PYY(25-36), [Ser³², Thr³⁶]PYY(25-36), [Ser³², Phe³⁶]PYY(25-36), [Lys³³, Asn³⁴]PYY(25-36), [Lys³³, Lys³⁵]PYY(25-36), [Lys³³, Thr³⁶]PYY(25-36), [Lys³³, Phe³⁶]PYY(25-36), [Asn³⁴, Lys³⁵]PYY(25-36), [Asn³⁴, Thr³⁶]PYY(25-36), [Asn³⁴, Phe³⁶]PYY(25-36), [Lys³⁵, Thr³⁶]PYY(25-36), or [Lys³⁵, Phe³⁶]PYY(25-36).

In some embodiments, the metabolic hormone is leptin or an analog, variant, or biologically active fragment thereof. In some embodiments, leptin or its variant or biologically active fragment thereof has at least 70% (e.g., at least 70%, 75%, 80%, 85%, 90%, 95%, 97%, 99%, or 100%) sequence identity to SEQ ID NO: 6. In some embodiments, leptin has the amino acid sequence set forth in SEQ ID NO: 6. In some embodiments, the analog of leptin is the recombinant analog metreleptin (MYALEPT®), which contains a polypeptide having the sequence set forth in SEQ ID NO: 9 and contains a disulfide bridge connecting amino acid residues 97 and 147. In some embodiments, the analog of leptin is a murine leptin analog, e.g., as described in Peters et al. Endocrinol. 148:2878-2885, 2007, which is hereby incorporated by reference in its entirety. In some embodiments, the methods described herein include administration of leptin or variant, analog, or biologically active fragment thereof in a dose of from about 2.5 μg to about 2.5 mg. In some embodiments, the methods described herein include administration of leptin or variant, analog, or biologically active fragment thereof in a dose of from about 10 μg to about 1 mg. In some embodiments, the methods described herein include administration of leptin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is amylin or a variant or biologically active fragment thereof. In some embodiments, amylin or its variant, analog, or biologically active fragment thereof has at least 70% (e.g., at least 70%, 75%, 80%, 85%, 90%, 95%, 97%, 99%, or 100%) sequence identity to SEQ ID NO: 7. In some embodiments, amylin has the amino acid sequence set forth in SEQ ID NO: 7. In some embodiments, the variant of amylin is pramlintide (SYMLIN®), which contains a polypeptide having to the sequence set forth in SEQ ID NO: 8. In some embodiments, the methods described herein include administration of amylin or variant, analog, or biologically active fragment thereof in a dose of from about 2.5 μg to about 2.5 mg. In some embodiments, the methods described herein include administration of amylin or variant, analog, or biologically active fragment thereof in a dose of from about 10 μg to about 1 mg. In some embodiments, the methods described herein include administration of amylin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is GLP-1 or an analog, variant, or biologically active fragment thereof. In some embodiments, GLP-1 or variant, analog, or biologically active fragment thereof has at least 70% (e.g., at least 70%, 75%, 80%, 85%, 90%, 95%, 97%, 99%, or 100%) sequence identity to SEQ ID NO: 3. In some embodiments, GLP-1 has the amino acid sequence set forth in SEQ ID NO: 3. In some embodiments, the GLP-1 fragment is GLP-1 (7-36) or variant, analog, or biologically active fragment thereof. In some embodiments, GLP-1 (7-36) or variant, analog, or biologically active fragment thereof has at least 70% (e.g., at least 70%, 75%, 80%, 85%, 90%, 95%, 97%, 99%, or 100%) sequence identity to SEQ ID NO: 4. In some embodiments, GLP-1 (7-36) has the amino acid sequence set forth in SEQ ID NO: 4. In some embodiments, the GLP-1 fragment is GLP-1 (7-37) or variant, analog, or biologically active fragment thereof. In some embodiments, GLP-1 (7-37) or variant, analog, or biologically active fragment thereof has at least 70% (e.g., at least 70%, 75%, 80%, 85%, 90%, 95%, 97%, or 100%) sequence identity to SEQ ID NO: 5. In some embodiments, GLP-1 (7-37) has the amino acid sequence set forth in SEQ ID NO: 5. In some embodiments, the methods described herein include administration of GLP-1 or variant, analog, or biologically active fragment thereof in a dose of from about 2.5 μg to about 2.5 mg. In some embodiments, the methods described herein include administration of GLP-1 or variant, analog, or biologically active fragment thereof in a dose of from about 10 μg to about 1 mg. In some embodiments, the methods described herein include administration of GLP-1 or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is calcitonin or an analog, variant, or biologically active fragment thereof. In some embodiments, calcitonin or variant, analog, or biologically active fragment thereof has at least 70% (e.g., 70%, 75%, 80%, 85%, 90%, 95%, 97%, 99%, or 100%) sequence identity to SEQ ID NO: 10. In some embodiments, calcitonin has the amino acid sequence set forth in SEQ ID NO: 10. In some embodiments, the methods described herein include administration of calcitonin or variant, analog, or biologically active fragment thereof in a dose of from about 2.5 μg to about 2.5 mg. In some embodiments, the methods described herein include administration of calcitonin or variant, analog, or biologically active fragment thereof in a dose of from about 10 μg to about 1 mg. In some embodiments, the methods described herein include administration of calcitonin or variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is insulin, or an analog (e.g., insulin aspart (NOVOLOG®), insulin glargine (LANTUS®), insulin lispro (LYUMJEV™), insulin glulisine (APIDRA®), or insulin detemir (LEVEMIR®), insulin degludec (TRESIBA®), NPH insulin (HUMULIN® N or NOVOLIN® N), a variant, or biologically active fragment thereof. In some embodiments, insulin or a variant, analog, or biologically active fragment thereof has at least 70% (e.g., at least 70%, 75%, 80%, 85%, 90%, 95%, 97%, 99%, or 100%) sequence identity to SEQ ID NO: 12. In some embodiments, insulin has the amino acid sequence set forth in SEQ ID NO: 12. In some embodiments, the analog of insulin is insulin aspart (NOVOLOG®), having an A chain with the sequence set forth in SEQ ID NO: 13 and a B chain with the sequence set forth in SEQ ID NO: 14. In some embodiments, the analog of insulin is insulin glargine (LANTUS®), having an A chain with sequence set forth in SEQ ID NO: 15 and a B chain with to the sequence set forth in SEQ ID NO: 16. In some embodiments, the analog of insulin is insulin lispro (LYUMJEV™), having an A chain with the sequence set forth in SEQ ID NO: 17 and a B chain with to the sequence set forth in SEQ ID NO: 18. In some embodiments, the analog of insulin is insulin glulisine (APIDRA®), having an A chain with the sequence set forth in SEQ ID NO: 19 and a B chain with to the sequence set forth in SEQ ID NO: 20. In some embodiments, the analog of insulin is insulin detemir (LEVEMIR®), having an A chain with to the sequence set forth in SEQ ID NO: 21 and a B chain with to the sequence set forth in SEQ ID NO: 22. In some embodiments, the methods herein described include the administration (e.g., topical) of insulin or a variant, analog, or biologically active fragment thereof in a dose of from about 2.5 μg to about 2.5 mg. In some embodiments, the methods herein described include the administration (e.g., topical) of insulin or a variant, analog, or biologically active fragment thereof in a dose of from about 10 μg to about 1 mg.

### Pharmaceutical Compositions and Routes of Administration

The metabolic hormones or variants or biologically active fragments thereof described herein can be formulated as pharmaceutical compositions for administration to human subjects in a biologically compatible form suitable for administration in vivo.

The compositions described herein may be administered to a subject (e.g., a human) in a variety of forms depending on the selected route of administration, as will be understood by those skilled in the art. The compositions described herein may be administered, for example, by any route that allows the composition (e.g., the metabolic) to reach the target receptor without substantially changing the concentration of the metabolic hormone in the blood of the subject. The composition may be administered, for example, intranasal, intrarectal, or topical GI routes.

In some embodiments, the compositions described herein are formulated for intranasal delivery. The intranasal composition may be formulated, e.g., as a spray, a semisolid, a particular, or in a lipid-based carrier. The formulation may be, e.g., a solution, a suspension, a powder, or a gel. Suitable intranasal formulations are described, e.g., in Marx et al. Drug Discov Dev 299-320, 2015, which is hereby incorporated by reference in its entirety. In some preferred embodiments, the compositions described herein are administered via inhalation, e.g., via nasal inhalation. An inhalable composition described herein may be provided as a liquid dosage form or dry powder dosage form. A dry powder composition may be, e.g., administered by inhalation as is or after reconstitution in a vehicle, e.g., saline (e.g., isotonic saline), phosphate-buffered saline, or water.

In some embodiments, the compositions described herein are formulated for topical administration to the GI tract. The topical composition may be formulated, e.g., as a GI patch. Suitable GI patch formulations are described, e.g., in Tao, et al Drug discovery Today 10:909-915, 2005, which is hereby incorporated by reference it its entirety.

In some embodiments, the compositions described herein are formulated for intrarectal delivery. The intrarectal composition may be formulated, e.g., as a suppository, an enema, an ointment, or a rectal foam. Suitable intrarectal formulations are described, e.g., in Hua Front. Pharmacol. 10:1196, 2019, which is hereby incorporated by reference in its entirety. Compositions for rectal administration may be in the form of suppositories containing a conventional suppository base, such as cocoa butter.

Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018.

The composition described herein may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

In some embodiments, the compositions include excipients that increase the time the metabolic hormone (e.g., PYY(3-36)) is in contact with the mucosa (e.g., nasal mucosa, GI mucosa, or rectal mucosa). The excipients may provide viscosity enhancement, encapsulation, and controlled release. Without being bound by theory, it is believed that increasing the contact time of the pharmaceutical formulation with the mucosa, leads to increased binding of the metabolic hormone to its receptor. Suitable excipients for viscosity enhancement include rheology modifiers which also may be mucoadhesive such as methylcellulose, hydroxyethylcellulose, hydroxypropylcellulose, alginic acid, polyvinylpyrrolidone, and sodium carboxymethylcellulose. Suitable excipients for modified release of the metabolic hormone in the mucosa include mucoadhesive permeation enhancers such as 23-lauryl ether, aprotinin, azone, benzalkonium chloride, cetylpyridinium chloride, cetyltrimethylammonium bromide, cyclodextrins, dextran sulfate, and lauric acid. Other suitable mucoadhesive polymers used for the compositions described herein include agarose, chitosan, gelatin, hyaluronic acid, gums (e.g. guar, hakea, xanthan, gellan, carrageenan, pectin, and sodium alginate), cellulose derivatives (e.g., CMC, thiolated CMC, sodium CMC, HEC, HPMC, MC, methylhydroxylethylcellulose), poly(acrylic acid)-based polymers (e.g., CP, PC, PAA, polyacrylates, poly(methylvinylether-co-methacrylic acid), poly(2-hydroxyethyl methacryalate), poly(alkylcyanoacryalate), poly(isohexylcyanocrylate), poly(isobutylcyanoacrylate), copolymer of acrylic acid and PEG, poly(N-2-hydroxypropyl methacrylamide), PHPMAm, polyoxyethylene, PVA, PVP, and other thiolated polymers; scleroglucan, PVA, steroidal detergents, non-ionic surfactants, laureth-9, sodium fusidate, included sodium lauryl, sodium laurate (e.g., pH 8.9), palmitoyl carnitine, lauric acid/propylene glycol vehicle, Brij 78, sodium deoxycholate, sodium lauryl sulfate, lecithin and PVP. See, e.g., International Journal of Pharmaceutics, Volume 53, Issue 3, 1 Aug. 1989, Pages 227-235.

A composition containing a metabolic hormone as described herein, can include one or more pharmaceutically acceptable excipients, such as propylene glycol, potassium sorbate, l-arginine, edetate disodium, monosodium phosphate, and polysorbate 20. Furthermore, the compositions described herein can include co-solvent stabilizers like propylene glycol or other suitable co-solvent stabilizers (e.g., lower molecular weight polyethylene glycols (PEG) such as PEG 200 and 400, glycerin, and ethanol. In some embodiments, compositions described herein include amino acid stabilizers like L-arginine or other suitable amino acid stabilizers (e.g., alanine, aspartic acid, glycine, lysine, proline, or methionine). In some embodiments, compositions described herein can include preservatives like potassium sorbate, or other suitable preservatives (e.g., ascorbic acid, benzyl alcohol, benzoic acid, citric acid, chlorobutanol, m-cresol, glutathione, methionine, methylparaben, propylparaben, sodium sulfite, parahydroxybenzoate esters (methylhydroxybenzoate and propylhydroxybenzoate), boric acid and borate salts, sorbic acid and other sorbate salts besides potassium, and phenolics). compositions described herein can include antioxidants such as edetate disodium or another suitable antioxidant (e.g., sodium formaldehyde sulphoxylate, butylated hydroxyanisole, and butylated hydroxytoluene). In some embodiments, the compositions described herein include buffers (e.g., acetate, carbonate, citrate, citrate-phosphate, glycine, HEPES, histidine, maleate, phosphate, succinate, tartrate, and triethanolamine (Tris)). In some embodiments, the compositions described herein can include surfactants, such as polysorbate 20 or other suitable surfactants (e.g., Poloxamer 188/407, polysorbate 40 or 80, or sodium lauryl sulfate).

In some embodiments, the pharmaceutical composition may be administered as in a unit dose form or as a dose per mass or weight of the patient from 0.01 ng/kg to 250 μg/kg (e.g., for a person of 75 kg body weight). For example, the dose of the metabolic hormone may be from 0.01 ng/kg to 0.1 ng/kg, e.g., 0.01 ng/kg, 0.02 ng/kg, 0.03 ng/kg, 0.04 ng/kg, 0.05 ng/kg, 0.06 ng/kg, 0.07 ng/kg, 0.08 ng/kg, 0.09 ng/kg, or 0.1 ng/kg, e.g., from 0.1 ng/kg to 1 ng/kg, e.g., 0.1 ng/kg, 0.2 ng/kg, 0.3 ng/kg, 0.4 ng/kg, 0.5 ng/kg, 0.6 ng/kg, 0.7 ng/kg, 0.8 ng/kg, 0.9 ng/kg, or 1 ng/kg, e.g., from 1 ng/kg to 10 ng/kg, e.g., 1 ng/kg, 2 ng/kg, 3 ng/kg, 4 ng/kg, 5 ng/kg, 6 ng/kg, 7 ng/kg, 8 ng/kg, 9 ng/kg, or 10 ng/kg, e.g., from 10 ng/kg to 100 ng/kg, e.g., 10 ng/kg, 20 ng/kg, 30 ng/kg, 40 ng/kg, 50 ng/kg, 60 μg/kg, 70 ng/kg, 80 ng/kg, 90 ng/kg, or 100 ng/kg, e.g., from 100 ng/kg to 1 μg/kg, e.g., 100 ng/kg, 200 ng/kg, 300 ng/kg, 400 ng/kg, 500 ng/kg, 600 ng/kg, 700 ng/kg, 800 ng/kg, 900 ng/kg, or 1 μg/kg, e.g., from 1 μg/kg to 10 μg/kg, e.g., 1 μg/kg, 2 μg/kg, 3 μg/kg, 4 μg/kg, 5 μg/kg, 6 μg/kg, 7 μg/kg, 8 μg/kg, 9 μg/kg, or 10 μg/kg, or e.g., from 10 μg/kg to 250 μg/kg, e.g., 10 μg/kg, 20 μg/kg, 30 μg/kg, 40 μg/kg, 50 μg/kg, 60 μg/kg, 70 μg/kg, 80 μg/kg, 90 ng/kg, 100 μg/kg, 110 μg/kg, 120 μg/kg, 130 μg/kg, 140 μg/kg, 150 μg/kg, 160 μg/kg, 170 μg/kg, 180 μg/kg, 190 g/kg, 200 μg/kg, 210 μg/kg, 220 μg/kg, 230 μg/kg, 240 μg/kg, or 250 μg/kg.

In general, the dosage of a pharmaceutical composition or the active agent (e.g., metabolic hormone, e.g., PYY (e.g., PYY(3-36)), GLP-1, leptin, amylin, insulin, or calcitonin, or a variant, analog, or biologically active fragment thereof) in a pharmaceutical composition may be in the range of from about 10 ng to about 200 μg per kg body weight, e.g., from about 100 ng to about 10 μg per kg body weight, or e.g., from about 100 ng to about 2.5 μg per kg body weight, e.g., a dose of about 100 ng, 200 ng, 300 ng, 400 ng, 500 ng, 600 ng, 700 ng, 800 ng, 900 ng, 1 μg, 2 μg, or 2.5 μg per kg body weight (e.g., for a person of 75 kg body weight).

Furthermore, it is understood that the dosage of an analog, variant, or biologically active fragment of the active agent may be administered as a molar equivalent amount of the active agent. The skilled artisan would understand, for example, that a metabolic hormone analog containing a posttranslational modification or a half-life extending moiety may require an increased (e.g., by 5%, 10%, 15%, 20%, 25%, 30%, 40%, 50%, 60%, 70%, 80%, 90%, 100%, 200%, 300%, or more) dosage than the dosage of the corresponding metabolic hormone without the posttranslational modification or half-life extending moiety.

The pharmaceutical composition may also be administered as a dose per mass or weight of the patient per unit day (e.g., 0.01 ng/kg/day to 250 μg/kg/day). In some embodiments, the pharmaceutical composition is administered at a dose from 10 ng/kg/day to 200 μg/kg/day (e.g., from 50 ng/kg/day to 100 μg/kg/day, from 100 ng/kg/day to 50 μg/kg/day, from 500 ng/kg/day to 1 μg/kg/day). In some embodiments, the pharmaceutical composition is administered at a dose from 100 ng/kg/day to 10 μg/kg/day (e.g., 100 ng/kg/day, 110 ng/kg/day, 120 ng/kg/day, 130 ng/kg/day, 140 ng/kg/day, 150 ng/kg/day, 160 ng/kg/day, 170 ng/kg/day, 180 ng/kg/day, 190 ng/kg/day, 200 ng/kg/day, 210 ng/kg/day, 220 ng/kg/day, 230 ng/kg/day, 240 ng/kg/day, 250 ng/kg/day, 260 ng/kg/day, 270 ng/kg/day, 280 ng/kg/day, 290 ng/kg/day, 300 ng/kg/day, 310 ng/kg/day, 320 ng/kg/day, 330 ng/kg/day, 340 ng/kg/day, 350 ng/kg/day, 360 ng/kg/day, 370 ng/kg/day, 380 ng/kg/day, 390 ng/kg/day, 400 ng/kg/day, 410 ng/kg/day, 420 ng/kg/day, 430 ng/kg/day, 440 ng/kg/day, 450 ng/kg/day, 460 ng/kg/day, 470 ng/kg/day, 480 ng/kg/day, 490 ng/kg/day, 500 ng/kg/day, 510 ng/kg/day, 520 ng/kg/day, 530 ng/kg/day, 540 ng/kg/day, 550 ng/kg/day, 560 ng/kg/day, 570 ng/kg/day, 580 ng/kg/day, 590 ng/kg/day, 600 ng/kg/day, 610 ng/kg/day, 620 ng/kg/day, 630 ng/kg/day, 640 ng/kg/day, 650 ng/kg/day, 660 ng/kg/day, 670 ng/kg/day, 680 ng/kg/day, 690 ng/kg/day, 700 ng/kg/day, 710 ng/kg/day, 720 ng/kg/day, 730 ng/kg/day, 740 ng/kg/day, 750 ng/kg/day, 760 ng/kg/day, 770 ng/kg/day, 780 ng/kg/day, 790 ng/kg/day, 800 ng/kg/day, 810 ng/kg/day, 820 ng/kg/day, 830 ng/kg/day, 840 ng/kg/day, 850 ng/kg/day, 860 ng/kg/day, 870 ng/kg/day, 880 ng/kg/day, 890 ng/kg/day, 900 ng/kg/day, 910 ng/kg/day, 920 ng/kg/day, 930 ng/kg/day, 940 ng/kg/day, 950 ng/kg/day, 960 ng/kg/day, 970 ng/kg/day, 980 ng/kg/day, 990 ng/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, or 10 μg/kg/day). In some embodiments, the pharmaceutical composition is administered at a dose from about 100 ng/kg/day to about 2.5 μg/kg/day (e.g., 100 ng/kg/day, 110 ng/kg/day, 120 ng/kg/day, 130 ng/kg/day, 140 ng/kg/day, 150 ng/kg/day, 160 ng/kg/day, 170 ng/kg/day, 180 ng/kg/day, 190 ng/kg/day, 200 ng/kg/day, 210 ng/kg/day, 220 ng/kg/day, 230 ng/kg/day, 240 ng/kg/day, 250 ng/kg/day, 260 ng/kg/day, 270 ng/kg/day, 280 ng/kg/day, 290 ng/kg/day, 300 ng/kg/day, 310 ng/kg/day, 320 ng/kg/day, 330 ng/kg/day, 340 ng/kg/day, 350 ng/kg/day, 360 ng/kg/day, 370 ng/kg/day, 380 ng/kg/day, 390 ng/kg/day, 400 ng/kg/day, 410 ng/kg/day, 420 ng/kg/day, 430 ng/kg/day, 440 ng/kg/day, 450 ng/kg/day, 460 ng/kg/day, 470 ng/kg/day, 480 ng/kg/day, 490 ng/kg/day, 500 ng/kg/day, 510 ng/kg/day, 520 ng/kg/day, 530 ng/kg/day, 540 ng/kg/day, 550 ng/kg/day, 560 ng/kg/day, 570 ng/kg/day, 580 ng/kg/day, 590 ng/kg/day, 600 ng/kg/day, 610 ng/kg/day, 620 ng/kg/day, 630 ng/kg/day, 640 ng/kg/day, 650 ng/kg/day, 660 ng/kg/day, 670 ng/kg/day, 680 ng/kg/day, 690 ng/kg/day, 700 ng/kg/day, 710 ng/kg/day, 720 ng/kg/day, 730 ng/kg/day, 740 ng/kg/day, 750 ng/kg/day, 760 ng/kg/day, 770 ng/kg/day, 780 ng/kg/day, 790 ng/kg/day, 800 ng/kg/day, 810 ng/kg/day, 820 ng/kg/day, 830 ng/kg/day, 840 ng/kg/day, 850 ng/kg/day, 860 ng/kg/day, 870 ng/kg/day, 880 ng/kg/day, 890 ng/kg/day, 900 ng/kg/day, 910 ng/kg/day, 920 ng/kg/day, 930 ng/kg/day, 940 ng/kg/day, 950 ng/kg/day, 960 ng/kg/day, 970 ng/kg/day, 980 ng/kg/day, 990 ng/kg/day, 1 μg/kg/day, 1.1 μg/kg/day, 1.2 μg/kg/day, 1.3 μg/kg/day, 1.4 μg/kg/day, 1.5 μg/kg/day, 1.6 μg/kg/day, 1.7 μg/kg/day, 1.8 μg/kg/day, 1.9 μg/kg/day, 2 μg/kg/day, 2.1 μg/kg/day, 2.2 μg/kg/day, 2.3 μg/kg/day, 2.4 μg/kg/day, or 2.5 μg/kg/day).

The dosage of the compositions (e.g., a composition including a metabolic hormone) described herein, can vary depending on many factors, such as the pharmacodynamic properties of the metabolic hormone, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition (e.g., a composition including a metabolic hormone) is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously. For example, a rectal formulation or GI patch may be present on the subject for a sustained amount of time (e.g., for at least 1 hour, 2 hours, 3 hours, 4 hours, 5 hours, 6 hours, 12 hours, 24 hours, 2 days, 3 days, 4 days, 5 days, 6 days, 7 days, 2 weeks, 3 weeks, 4 weeks, 2 months, 3 months, or longer).

The pharmaceutical compositions described herein (e.g., containing a metabolic hormone) may be provided in a kit that includes the pharmaceutical composition (e.g., in a container) and instructions for use thereof. The kit may contain one or more containers, in which each container contains a different composition of the invention. The instructions enclosed with the kit may be used to instruct a user to perform a method as described herein.

The methods described herein include administration of a metabolic hormone (e.g., PYY, PYY(3-36), leptin, amylin, insulin, GLP-1, or an analog, variant, or biologically active fragment thereof locally to the rectum, nasal cavity, or GI tract of a subject, wherein the local administration does not produce substantial change to the level of the metabolic hormone in the blood and/or plasma of the subject. In general, the metabolic hormone level in the blood and/or plasma of the subject does not increase more than up to 10% (e.g., 9%, 8%, 7%, 6%, 5%, 4%, 3%, 2%, 1%, or less) of the pre-administration level of metabolic hormone. In some embodiments, the blood and/or plasma level of PYY(3-36) does not substantially surpass pre-prandial levels of from about 15 μmol/l to about 25 μmol/l as reported in Batterham et al, Cell Metabolism, 4:223-233, 2006, herein incorporated by reference, in its entirety, after administration (e.g., topical) of PYY(3-36). In some embodiments, the blood and/or plasma level of leptin does not substantially surpass pre-prandial levels of from about 5 ng/ml to about 35 ng/ml as reported in Considine et al, N. Engl. J. Med. 334:292-295, 1996, herein incorporated by reference in its entirety, after administration (e.g., topical) of leptin. In some embodiments, the blood and/or plasma level of amylin does not substantially surpass pre-prandial levels of about 20 μmol/l as reported in Cooper et al, Hypertension, 26:460-464, 1995, herein incorporated by reference in its entirety, after administration (e.g., topical) of amylin.

### Indications

The methods described herein include the administration of a metabolic hormone for the induction of satiety and/or treatment of metabolic syndrome, diabetes, obesity, or any obesity-related disorder.

In some embodiments, the subject in need of treatment has been diagnosed with or it is at risk of a metabolic syndrome, obesity, an obesity-related disorder, diabetes, fatty liver disease, nonalcoholic steatohepatitis, chronic kidney disease, polycystic ovary syndrome, cardiovascular disease, obstructive sleep apnea, retinopathy, peripheral vascular disease, peripheral artery disease, and neuropathy (e.g., diabetic neuropathy). In some embodiments, the methods are used for maintenance of weight or prevention of weight gain.

Sequences

PYY
SEQ ID NO: 1
MVFVRRPWPALTTVLLALLVCLGALVDAYPIKPEAPREDASPEELNRYYASLRHYLNLVTRQRYGKRDGPDT
LLSKTFFPDGEDRPVRSRSEGPDLW
PYY(3-36)
SEQ ID NO: 2
IKPEAPGEDASPEELNRYYASLRHYLNLVTRQRY
GLP-1
SEQ ID NO: 3
MKSIYFVAGLFVMLVQGSWQRSLQDTEEKSRSFSASQADPLSDPDQMNEDKRHSQGTFTSDYSKYLDSRR
AQDFVQWLMNTKRNRNNIAKRHDEFERHAEGTFTSDVSSYLEGQAAKEFIAWLVKGRGRRDFPEEVAIVEEL
GRRHADGSFSDEMNTILDNLAARDFINWLIQTKITDRK
GLP-1(7-36)
SEQ ID NO: 4
HAEGTFTSDVSSYLEGQAAKEFIAWLVKGR
GLP-1(7-37)
SEQ ID NO: 5
HAEGTFTSDVSSYLEGQAAKEFIAWLVKGRG
Leptin
SEQ ID NO: 6
MHWGTLCGFLWLWPYLFYVQAVPIQKVQDDTKTLIKTIVTRINDISHTQSVSSKQKVTGLDFIPGLHPILTLSKM
DQTLAVYQQILTSMPSRNVIQISNDLENLRDLLHVLAFSKSCHLPWASGLETLDSLGGVLEASGYSTEVVALS
RLQGSLQDMLWQLDLSPGC
Amylin
SEQ ID NO: 7
MGILKLQVFLIVLSVALNHLKATPIESHQVEKRKCNTATCATQRLANFLVHSSNNFGAILSSTNVGSNTYGKRN
AVEVLKREPLNYLPL
Pramlintide
SEQ ID NO: 8
KCNTATCATQRLANFLVHSSNNFGPILPPTNVGSNTY
Metreleptin
SEQ ID NO: 9
MVPIQKVQDDTKTLIKTIVTRINDISHTQSVSSKQKVTGLDFIPGLHPILTLSKMDQTLAVYQQILTSMPSRNVIQI
SNDLENLRDLLHVLAFSKSCHLPWASGLETLDSLGGVLEASGYSTEVVALSRLQGSLQDMLWQLDLSPGC
(Disulfide bridge: 97-147)
Calcitonin
SEQ ID NO: 10
CGNLSTCMLGTYTQDFNKFHTFPQTAIGVGAP
[Pro34]PYY
SEQ ID NO: 11
YPIKPEAPGEDASPEELNRYYASLRHYLNLVTRPRY
Insulin
SEQ ID NO: 12
MALWMRLLPLLALLALWGPDPAAAFVNQHLCGSHLVEALYLVCGERGFFYTPKTRREAEDLQVGQVELGGG
PGAGSLQPLALEGSLQKRGIVEQCCTSICSLYQLENYCN
Insulin Aspart
A chain
SEQ ID NO: 13
GIVEQCCTSICSLYQLENYCN
Insulin Aspart
B chain
SEQ ID NO: 14
FVNQHLCGSHLVEALYLVCGERGFFYTDKT
Insulin Glargine
A chain
SEQ ID NO: 15
GIVEQCCTSICSLYQLENYCG
Insulin Glargine
B chain
SEQ ID NO: 16
FVNQHLCGSHLVEALYLVCGERGFFYTPKTRR
Insulin Lispro
A chain
SEQ ID NO: 17
GIVEQCCTSICSLYQLENYCN
Insulin Lispro
B chain
SEQ ID NO: 18
FVNQHLCGSHLVEALYLVCGERGFFYTKPT
Insulin Glulisine
A chain
SEQ ID NO: 19
GIVEQCCTSICSLYQLENYCN
Insulin Glulisine
B chain
SEQ ID NO: 20
FVKQHLCGSHLVEALYLVCGERGFFYTPET
Insulin Detemir
A chain
SEQ ID NO: 21
GIVEQCCTSICSLYQLENYCN
Insulin Detemir
B chain
SEQ ID NO: 22
FVNQHLCGSHLVEALYLVCGERGFFYTPK

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method for inducing satiety or treating a disease or disorder selected from a metabolic syndrome, obesity, an obesity-related disorder, diabetes, fatty liver disease, nonalcoholic steatohepatitis, chronic kidney disease, polycystic ovary syndrome, cardiovascular disease, obstructive sleep apnea, retinopathy, peripheral vascular disease, peripheral artery disease, and neuropathy in a subject in need thereof, the method comprising administering to the subject a composition comprising an agent selected from Peptide YY (PYY), glucagon-like peptide 1 (GLP-1), leptin, amylin, insulin, and calcitonin, or an analog, variant, or biologically active fragment thereof, wherein the composition is administered intranasally, topically to the gastrointestinal (GI) tract, or intrarectally, and wherein the composition provides treatment without substantially changing the concentration of the agent in the blood of the subject.

2. The method of claim 1, wherein the composition is administered intranasally.

3. The method of claim 2, wherein the composition is formulated as a spray, a semisolid, a particulate, or in a lipid-based carrier.

4. The method of claim 1, wherein the composition is administered topically to the GI tract.

5. The method of claim 4, wherein the composition is formulated as a GI patch.

6. The method of claim 1, wherein the composition is administered intrarectally.

7. The method of claim 6, wherein the composition is formulated as a suppository.

8. The method of claim 1, wherein the dose of the agent is from 1 ng to 20 mg per 100 kg body weight.

9. The method of claim 8, wherein the dose of the agent is from 1 ng to 1 μg per 100 kg body weight.

10. The method of claim 8, wherein the dose of the agent is from 1 μg to 1 mg per 100 kg body weight.

11. The method of claim 8, wherein the dose of the agent is from 1 mg to 20 mg per 100 kg body weight.

12. The method of claim 1, wherein the composition comprises PYY.

13. The method of claim 12, wherein the PYY is PYY(3-36).

14. The method of claim 1, wherein the composition comprises GLP-1.

15. The method of claim 1, wherein the composition comprises leptin.

16. The method of claim 1, wherein the composition comprises amylin.

17. The method of claim 1, wherein the composition comprises insulin.

18. The method of claim 1, wherein the composition comprises calcitonin.


SEQUENCE LISTING (22 sequences)

SEQ ID NO: 1 - protein, 97 aa, Homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 2 - protein, 34 aa, Homo sapiens
IKPEAPGEDASPEELNRYYASLRHYLNLVTRQRY

SEQ ID NO: 3 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 4 - protein, 30 aa, Homo sapiens
HAEGTFTSDVSSYLEGQAAKEFIAWLVKGR

SEQ ID NO: 5 - protein, 31 aa, Homo sapiens
HAEGTFTSDVSSYLEGQAAKEFIAWLVKGRG

SEQ ID NO: 6 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 7 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 8 - protein, 37 aa, synthetic construct
KCNTATCATQRLANFLVHSSNNFGPILPPTNVGSNTY

SEQ ID NO: 9 - protein, 147 aa, synthetic construct
  DISULFID 97..147: note = intrachain disulfide bond
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 10 - protein, 32 aa, Homo sapiens
CGNLSTCMLGTYTQDFNKFHTFPQTAIGVGAP

SEQ ID NO: 11 - protein, 36 aa, synthetic construct
YPIKPEAPGEDASPEELNRYYASLRHYLNLVTRPRY

SEQ ID NO: 12 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 13 - protein, 21 aa, synthetic construct
GIVEQCCTSICSLYQLENYCN

SEQ ID NO: 14 - protein, 30 aa, synthetic construct
FVNQHLCGSHLVEALYLVCGERGFFYTDKT

SEQ ID NO: 15 - protein, 21 aa, synthetic construct
GIVEQCCTSICSLYQLENYCG

SEQ ID NO: 16 - protein, 32 aa, synthetic construct
FVNQHLCGSHLVEALYLVCGERGFFYTPKTRR

SEQ ID NO: 17 - protein, 21 aa, synthetic construct
GIVEQCCTSICSLYQLENYCN

SEQ ID NO: 18 - protein, 30 aa, synthetic construct
FVNQHLCGSHLVEALYLVCGERGFFYTKPT

SEQ ID NO: 19 - protein, 21 aa, synthetic construct
GIVEQCCTSICSLYQLENYCN

SEQ ID NO: 20 - protein, 30 aa, synthetic construct
FVKQHLCGSHLVEALYLVCGERGFFYTPET

SEQ ID NO: 21 - protein, 21 aa, synthetic construct
GIVEQCCTSICSLYQLENYCN

SEQ ID NO: 22 - protein, 29 aa, synthetic construct
FVNQHLCGSHLVEALYLVCGERGFFYTPK